(19) United States
(12) Patent Application Publication
(10) Pub. No.: US 2004/0184951 A1
(43) Pub. Date: Sep. 23, 2004

(54) Method for sanitizing the water circuits of dental units and a dental unit implementing the method

(75) Inventor: Franco Castellini, Bologna (IT)

Correspondence Address: Richard J. Minnich, Esq., Fay, Sharpe, Fagan, Minnich & McKee, LLP, Seventh Floor, 1100 Superior Avenue, Cleveland, OH, 44114-2518, US

(73) Assignee: CASTELLINI S.p.A.

(21) Appl. No.: 10/392,087

(22) Filed: Mar. 19, 2003

(51) Int. Cl. A01N 1/00; A61L 9/00

(52) U.S. Cl. 422/28; 422/1; 422/3

(57) ABSTRACT

A method for sanitizing water circuits (1) of dental units (100) comprising dental handpieces (2) and a water circuit (1) for supplying fluid from a main supply (4) through corresponding circuit legs or branches (1a, 1b, 1c, 1s), the method comprising at least the following steps: introducing the disinfectant/sterilizing liquid in the water circuit (1) for a predetermined length of time and draining the disinfectant/sterilizing liquid out of the circuit through the circuit legs or branches (1a, 1b, 1c, 1s). The step of introducing the disinfectant/sterilizing liquid is divided into two or more sub-steps, each consisting of an inflow of a predetermined quantity or dose of the disinfectant/sterilizing liquid, alternated with steps of waiting for the next inflow.

### BACKGROUND OF THE INVENTION

The present invention relates to a method for sanitizing the water circuits of dental units and to a dental unit implementing the method.

In the manufacturing of dental equipment, one of the fundamental parts, if not the “heart”, of a dental unit is its water and air circuit, where the water line supplies fluids used by dental equipment and patients (water or physiological saline for tumblers and handpieces), or consumer units (swilling water for the spittoon), while the air line is used for certain items of equipment (air spray handpieces, cooling air and driving air).

With increases in general standards of hygiene and with dental apparatus and equipment becoming more and more “fragile”, several design solutions have been found for the water and air circuits of dental units, not only to guarantee their efficient operation and durability but also to maintain the sterility of the conduits during patient treatments. Considering that the basic structure of these fluid circuits comprises a first main line supplying water from the mains, and a second main line supplying air from an external source (compressor), each of which has a plurality of branches leading to the operating and accessory equipment of the dental unit, different systems have been designed on the basis of different methods aimed at improving the functioning and disinfection of these fluid lines or parts of them.

In particular, the present specification focuses attention on the water line which is disinfected according to two different methods, one with a continuous cycle and the other with a discontinuous cycle, and both requiring additional devices to be fitted to the basic structure of the circuit.

The present specification is concerned in particular with the solutions based on the discontinuous disinfection/sterilization cycle. In this type of cycle, as disclosed in patent publications EP-111.249 and EP-317.521 (the latter being by the present Applicant), the mains water supply is shut off, and a dedicated branch equipped with an independent tank is used to feed sterilizing liquid into the conduits that supply water to the handpieces.

After a preset time, depending on the quality of disinfection/sterilization required and the properties of the sterilizing liquid, the line is opened again and the sterilizing liquid drained out.

The drainage of the sterilizing liquid is performed by flushing water (or a user fluid) supplied by the main line (or by a dedicated line) and opening the control valves on the handpieces so that the water or user fluid expels the sterilizing liquid, rinses the water line and flows out into an appropriate drain.

Although this method, which has been used on dental units for some time, has proved to be very effective and practical, the Applicant, in line with a policy of continual improvement of dental unit sanitizing procedures, has continued to conduct numerous studies and research projects which have revealed that the sanitizing product, if supplied in a single, predetermined dose and held in the circuit for the entire contact period, tends to deteriorate, thus locally reducing the effect of the sanitizing treatment on account of a loss of effective product concentration in certain parts of the system.

The aim of the present invention is to overcome the above mentioned disadvantages by providing a method for sterilizing/disinfecting the water circuit of a dental unit such as to optimize the sanitizing action of the disinfectant/sterilizing liquid by keeping the liquid at a constantly high level of effectiveness at all times and in all parts of the circuit.

### SUMMARY OF THE INVENTION

The above mentioned aim is achieved in a method for sanitizing water circuits of dental units comprising dental handpieces and a water circuit for supplying fluid from a main supply through corresponding circuit legs or branches, the method comprising at least the following steps: introducing the disinfectant/sterilizing liquid in the water circuit for a predetermined length of time and draining the disinfectant/sterilizing liquid out of the circuit through the circuit legs or branches; the step of introducing the disinfectant/sterilizing liquid being divided into two or more sub-steps, each consisting of an inflow of a predetermined quantity or dose of the disinfectant/sterilizing liquid, alternated with steps of waiting for the next inflow.

### BRIEF DESCRIPTION OF THE DRAWINGS

The technical characteristics of the invention, with reference to the above aims, are clearly described in the claims below and its advantages are apparent from the detailed description which follows, with reference to the accompanying drawings which illustrate a preferred embodiment of the invention provided merely by way of example without restricting the scope of the inventive concept, and in which:

FIG. 1 shows a diagram representing the water circuit of a dental unit that implements the method according to the present invention for sanitizing the water circuit itself;

FIG. 2 shows a graph representing the effects obtained with the method according to the present invention.

### DESCRIPTION OF THE PREFERRED EMBODIMENTS

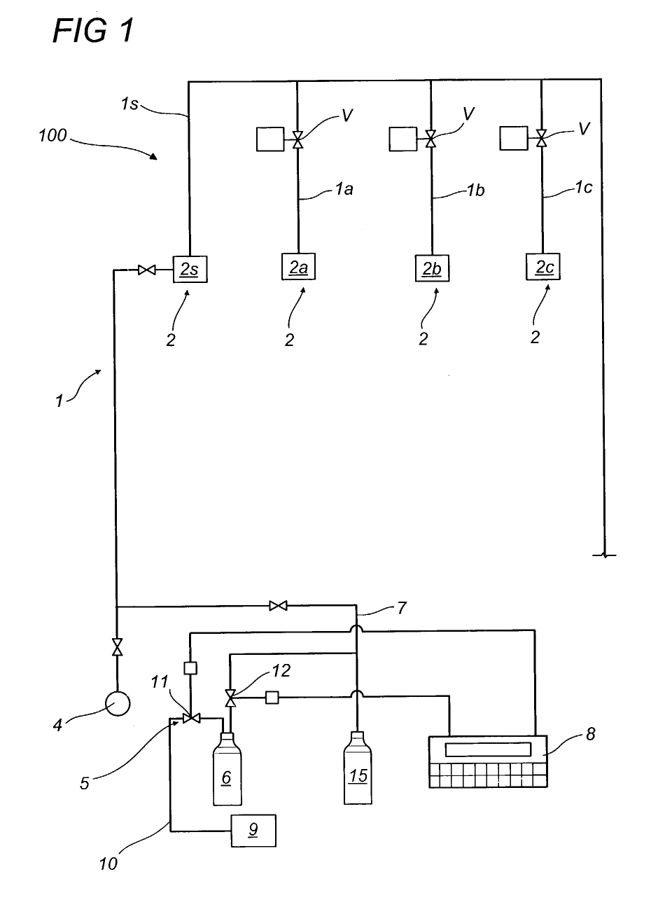

With reference to the accompanying drawings, in particular FIG. 1, the method according to the invention is used to sanitize the water circuit 1 of a dental unit 100.

The dental unit 100 is of the type comprising, at least insofar as is relevant to the present invention, medical instruments such as dental handpieces 2 (represented as a block in the drawings) equipped with shutoff valves V, and a water circuit 1 for feeding a fluid from a main supply 4 (water) or from an independent circuit leg that supplies sterile liquid through a suitable container 15.

The handpieces 2 (which may be, for example, a micromotor 2a, a turbine 2b, an ablator 2c and a syringe 2s) are supplied through corresponding legs or branches 1a, 1b, 1c, 1s of the circuit 1, which are equipped with the valves V.

The method according to the invention comprises at least the following steps:

introducing a disinfectant/sterilizing liquid in the water circuit 1 for a predetermined length of time;

draining the disinfectant/sterilizing liquid out of the circuit 1 through the circuit legs or branches 1a, 1b, 1c, 1s.

Obviously and as is well within the knowledge of experts in the trade familiar with methods of this kind, the valves V are opened and closed during these steps in order to allow the fluids to flow into the water circuit 1, be held there and then drained out.

In this particular case, the step of introducing the disinfectant/sterilizing liquid is divided into two or more sub-steps, each consisting of an inflow of a predetermined quantity or dose of the disinfectant/sterilizing liquid, alternated with steps of waiting for the next inflow of the liquid.

In other words, each of these sub-steps of disinfectant/sterilizing liquid inflow is such that the predetermined quantity or dose of the liquid is sufficient to renew the liquid (by completely filling) the water circuit 1, while the waiting time periods between one inflow and the next depends on the time the sanitizing product remains active before it starts losing its sanitizing charge. Thus, the total contact time, consisting of the time taken for the inflow steps added to the time periods of the steps of waiting between one inflow and another, is a function of the optimum time periods to sterilize/sanitize the circuit 1 and its branches.

Evidently, when a new dose of disinfectant/sterilizing liquid flows in, the dose already inside the water circuit 1 is flushed out of the circuit branches 1a, 1b, 1c, 1s.

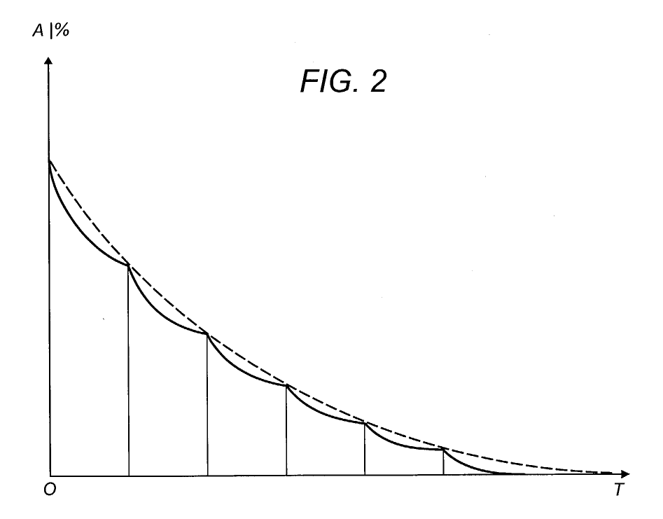

The sanitizing effect of the successive inflows of disinfectant/sterilizing liquid is illustrated purely by way of example in the graph of FIG. 2, where the x-axis represents the time T and the y-axis the percentage of polluting agents Al present in the water circuit. A comparison of the sanitizing performance of a traditional cycle (represented by the dashed line) with that of the new cycle, divided into sub-steps, according to the present invention (represented by the continuous line) shows the lafter's greater effectiveness in destroying the polluting agents per unit time, with obvious advantages in terms of overall water circuit hygiene.

The aforementioned sub-steps of feeding a certain quantity of disinfectant/sterilizing liquid are performed by feed means 5 acting on an independent container 6 of the disinfectant/sterilizing liquid connected to the water circuit 1 through a first independent circuit branch 7.

In the embodiment illustrated, the dental unit 100 may be equipped with a microprocessor unit 8 that controls the main and accessory functions of the dental unit 100.

The microprocessor unit 8 can be advantageously used to program the feed means 5 so that the disinfectant/sterilizing liquid is fed into the circuit in a predetermined, optimum dose during each inflow sub-step and left to take action for an optimum length of waiting time before the next dose is fed in.

The method according to the invention may comprise a further step of heating the disinfectant/sterilizing liquid just before it is fed into the water circuit 1: this is to further improve and speed up the biocidal action of the disinfectant/sterilizing liquid.

The dental unit that implements the method described above therefore comprises the above mentioned means 5 for the controlled infeed of the disinfectant/sterilizing liquid acting on a container 6 in which the liquid is stored and designed to supply the water circuit 1 with disinfectant/sterilizing liquid in a plurality of predetermined doses in such a way as to provide an active sanitizing action.

As mentioned above, the dental unit 100 comprises a microprocessor unit 8 that controls the main and accessory functions of the dental unit 100, the microprocessor unit 8 controlling and activating the feed means 5 in such a way as to alternate the steps of feeding the plurality of doses of disinfectant/sterilizing liquid into the water circuit 1 with steps of stopping the flow of the liquid.

The dental unit 100 further comprises an air circuit 9 for supplying air used by the dental unit 100 in a well-known manner for some of its main and accessory functions. The air circuit 9 is illustrated as a block and has branches connected with the parts relevant to the present invention purely by way of non-restrictive example.

The feed means 5 comprise a branch 10 of the air circuit 9. The circuit branch 10 is located in the container 6 of the disinfectant/sterilizing liquid and is activated by a shutoff element 11 (for example a valve) controlled by the microprocessor unit 8 so as to allow the doses of the disinfectant/sterilizing liquid to flow into the first independent branch 7.

The first independent branch 7 is equipped with a valve element 12 designed to stop and start the flow of disinfectant/sterilizing liquid from the container 6 and, by activating the shutoff element 11, the flow of air through the circuit branch 10. In practice, the valve element 12 and the shutoff element 11 are switched on and off in synchrony with each other by the microprocessor unit 8 in order to obtain the correct liquid inflow and hold steps according to the programmed doses and time intervals.

The method and dental unit as described above therefore achieve the preset aims by allowing inflows and outflows of successive doses of disinfectant/sterilizing liquid to be specifically programmed in such a way as to destroy polluting agents quickly and at a constant rate without necessitating more time than traditional cycles to accomplish a complete cycle of sure sterilization/disinfection.

The invention described can be subject to modifications and variations without thereby departing from the scope of the inventive concept. Moreover, all the details of the invention may be substituted by technically equivalent elements.

What is claimed is:

1. A method for sanitizing water circuits (1) of dental units comprising medical instruments, such as dental handpieces (2), and a water circuit (1) for supplying liquid from a main supply (4) through corresponding circuit legs or branches (1a, 1b, 1c, 1s); the method comprising at least the following steps: introducing the disinfectant/sterilizing liquid in the water circuit (1) for a predetermined length of time and draining the disinfectant/sterilizing liquid out of the circuit through the circuit legs or branches (1a, 1b, 1c, 1s), wherein the step of introducing the disinfectant/sterilizing liquid is divided into two or more sub-steps, each consisting of an inflow of a predetermined quantity or dose of the disinfectant/sterilizing liquid, alternated with steps of waiting for the next inflow.

2. The method according to claim 1, wherein the quantity or dose of disinfectant/sterilizing liquid flowing in during each of the sub-steps is predetermined and sufficient to completely fill the water circuit (1) and renew the liquid in it, the waiting time periods between one inflow and the next being such that the disinfectant/sterilizing liquid in the water circuit (1) is maintained at an effective concentration.

3. The method according to claim 1, wherein the sub-steps of feeding a certain quantity of disinfectant/sterilizing liquid are performed by feed means (5) acting on an independent container (6) of the disinfectant/sterilizing liquid connected to the water circuit (1) through a first independent circuit branch (7).

4. The method according to claim 1, where the dental unit (100) is equipped with a microprocessor unit (8) that controls the main and accessory functions of the dental unit (100), wherein the sub-steps of feeding in the doses of disinfectant/sterilizing liquid are performed by feed means (5) controlled by the microprocessor unit (8) so that the disinfectant/sterilizing liquid is fed into the circuit for a predetermined inflow time and left to take action in the circuit for a predetermined length of waiting time before the next dose is fed in.

5. The method according to claim 1, wherein the step of introducing the disinfectant/sterilizing liquid comprises a step of heating the disinfectant/sterilizing liquid flowing into the water circuit (1).

6. The method according to claim 1, wherein each sub-step of feeding a dose of disinfectant/sterilizing liquid is performed at the same time as a step of draining out the dose previously fed into the water circuit (1).

7. A dental unit comprising at least one main water circuit (1) for supplying a liquid to each handpiece (2) or accessory item of dental equipment present on the dental unit (100), and at least one independent circuit branch (7) connected to the water circuit (1) and supplying the water circuit (1) with at least one disinfectant/sterilizing liquid from a container (6), wherein the dental unit (100) further comprises means (5) for the controlled infeed of the disinfectant/sterilizing liquid acting on the container (6) and designed to supply the water circuit (1) with disinfectant/sterilizing liquid in a plurality of predetermined successive doses in such a way as to enable each dose of disinfectant/sterilizing liquid to remain in the circuit (1) for as long as it remains active.

8. The dental unit according to claim 7, where the dental unit (100) comprises a microprocessor unit (8) to control the main and accessory of the dental unit (100), wherein the disinfectant/sterilizing liquid feed means (5) are controlled by the microprocessor unit (8) in such a way as to alternate the steps of feeding the plurality of doses of disinfectant/sterilizing liquid into the water circuit (1) with steps of stopping the flow of the liquid.

9. The dental unit according to claims 7 and 8, where the dental unit (100) has an air supply circuit (9) used for its main and accessory functions, wherein the feed means (5) comprise a branch (10) of the air circuit (9) that is located in the container (6) of the disinfectant/sterilizing liquid and activated by a shutoff element (11) controlled by the microprocessor unit (8) so as to allow the doses of the disinfectant/sterilizing liquid to flow into the first independent branch (7).

10. The dental unit according to claim 9, wherein the first independent branch (7) is equipped with valve means (12) designed to stop and start the flow of disinfectant/sterilizing liquid from the container (6) and, by activating the shutoff element (11), the flow of air through the air circuit branch (10).

11. The dental unit according to claim 10, wherein the valve means (12) and the shutoff element (11) can be switched on and off by the microprocessor unit (8).
